(19) United States
(12) Patent Application Publication
(10) Pub. No.: US 2022/0176371 A1
(43) Pub. Date: Jun. 9, 2022

(54) APPARATUSES AND SAMPLE SWABS FOR PERFORMING RAPID DIAGNOSTIC TESTS

(71) Applicant: Detect, Inc., Guilford, CT (US)

(72) Inventors: Todd Roswech, Ivoryton, CT (US); Matthew Dyer, Spring, TX (US)

(73) Assignee: Detect, Inc., Guilford, CT (US)

(21) Appl. No.: 17/540,667

(22) Filed: Dec. 2, 2021

Related U.S. Application Data
(60) Provisional application No. 63/121,746, filed on Dec. 4, 2020.

(51) Int. Cl. B01L 3/00; B01L 7/00; C12Q 1/686; G01N 33/543

(52) U.S. Cl. CPC B01L 3/5029; B01L 7/525; C12Q 1/686; G01N 33/54388; B01L 2300/069; G01N 2800/26; B01L 2200/0689; B01L 2200/16; B01L 2300/044; B01L 2200/141; B01L 2300/1827; B01L 2300/0825

(57) ABSTRACT

Diagnostic devices for performing diagnostic tests are provided, as well as methods that utilize the diagnostic devices, methods for manufacturing the diagnostic devices, and test kits for performing the diagnostic tests. The diagnostic devices may include a sample chamber; a fluid chamber connected to the sample chamber by a conduit; and a test chamber separated from the sample chamber by a breakable seal. A movable liner may form a portion of an inner surface of the sample chamber. The sample chamber may be configured to receive a sample swab, which may move in a first direction when being inserted in the sample chamber to an inserted position, and which may to move in a second direction to cause the breakable seal to break. Movement of the sample swab in the second direction may cause the liner to move in the second direction to break the breakable seal.

### CROSS-REFERENCE TO RELATED APPLICATIONS

The present application claims the benefit of priority of U.S. Provisional Application No. 63/121,746 filed Dec. 4, 2020, entitled “APPARATUSES AND SAMPLE SWABS FOR PERFORMING RAPID DIAGNOSTIC TESTS” (Attorney Docket No. H0966.70046US00), the entire contents of which is incorporated by reference herein.

### FIELD

The technology of the present invention relates generally to test apparatuses, test kits, methods of using the test apparatuses and/or the test kits to perform rapid diagnostic tests to detect the presence of one or more target nucleic-acid sequences, and methods of making the test apparatuses and/or the test kits.

### BACKGROUND

The ability to rapidly diagnose diseases—particularly highly communicable infectious diseases—is critical to preserving human health through early detection and containment of the infectious diseases until reliable preventive measures (e.g., vaccines) and/or medicinal treatments or cures are developed. Rapid testing is critical to determining infected individuals quickly and minimizing their interactions with others, in order to minimize the spread of the diseases. As one example, the high level of contagiousness, the high mortality rate, and the lack of an early treatment or vaccine for the coronavirus disease 2019 (COVID-19) have resulted in a pandemic that has already infected millions and killed hundreds of thousands of people. The existence of rapid, accurate diagnostic tests, useable for detecting COVID-19 as well as other diseases, could allow individuals infected with a disease to be quickly identified and isolated, which could assist with containment of the disease. In the absence of such diagnostic tests, diseases such as COVID-19 may spread unchecked throughout communities.

### SUMMARY

Provided herein are apparatuses and techniques for performing diagnostic tests useful for detecting one or more pathogens by detecting one or more target nucleic-acid sequences corresponding to the pathogens. The apparatuses and techniques described herein enable a diagnostic test to be self-administrable by a subject to be tested, and the diagnostic test may be performed in a point-of-care (POC) setting or home setting by a lay person without specialized equipment and without training in laboratory procedures. In some embodiments, the apparatuses and techniques described herein may enable rapid diagnostic tests to be performed in less than one hour without sacrificing accuracy. That is, in some embodiments through use of, e.g., isothermal amplification methods, the rapid diagnostic tests enabled by the apparatuses and techniques described herein may provide a diagnosis having an accuracy on par with the accuracy of typical PCR tests in less than one hour.

According to an aspect of the present technology, a test apparatus for performing a rapid diagnostic test is provided. The apparatus may be comprised of: a sample chamber; a fluid chamber connected to the sample chamber by a conduit; and a test chamber separated from the sample chamber by a breakable first seal. The apparatus may further be comprised of a movable liner forming a portion of an inner surface of the sample chamber.

In some embodiments of this aspect, the apparatus may further be comprised of a sample swab for collecting a sample from a subject to be tested. The sample swab may be configured to be inserted into an opening of the sample chamber and to seal the sample chamber with a slidable seal. The sample swab may be comprised of: a swab element, and a handle comprised of a seal portion configured to slide along the inner surface of the sample chamber to slidably seal of an internal cavity of the sample chamber. The seal portion of the handle may be comprised of a gasket configured to press against and slide along the inner surface of the sample chamber to slidably seal the sample chamber.

In some embodiments of this aspect, the apparatus may further be comprised of a burstable capsule containing a first fluid. As described herein, the sample swab may be configured to cause directly, in some embodiments, or indirectly, in some embodiments, the capsule to burst when the sample swab is inserted in the sample chamber to an inserted position, such that the first fluid is permitted to form a sample solution in the sample chamber by interaction with a sample carried by the swab element of the sample swab. In some embodiments, the sample swab may be configured to be moved in a first direction to insert the swab element of the sample swab into the sample chamber, and to be moved from an inserted position in the sample chamber in a second direction, different from the first direction, to break the first seal. The liner may be configured to move with the sample swab in the second direction to break the first seal.

In some embodiments of this aspect, the fluid chamber may be separated from the sample chamber by a breakable second seal. The liner may be configured to move with the sample swab in the second direction to break the second seal. In some embodiments, the liner may be configured to break the second seal before breaking the first seal. The sample swab may be comprised of a handle with markings indicating at least one insertion position of the sample swab when the sample swab is inserted in the sample chamber.

In some embodiments of this aspect, the sample swab may be configured to extend into the internal cavity of the sample chamber to contact and burst the capsule. In some embodiments, the capsule may be disposed in the fluid chamber, the fluid chamber may be comprised of a crusher configured to move in the fluid chamber, and the crusher may be configured to be moved by a contact portion of the sample swab to burst the capsule, when the sample swab is inserted to the inserted position in the sample chamber.

In some embodiments of this aspect, the test chamber may be comprised of a lateral-flow assay (LFA) strip. When movement of the sample swab causes the first seal to break, the sample solution in the sample chamber is permitted to enter the test chamber. In some embodiments, the apparatus may be further comprised of a heater configured to heat the sample chamber.

According to another aspect of the present technology, a test apparatus for performing a rapid diagnostic test is provided. The apparatus may be comprised of: a first chamber; a second chamber separated from the first chamber by a breakable first seal; a third chamber separated from the first chamber by a breakable second seal; and a movable liner forming a portion of an inner surface of the first chamber, wherein the liner is comprised of one of or both of the first and second seals. The first seal and the second seal may, in some instances, be connected to each other.

In some embodiments of this aspect, the apparatus may further be comprised of a sample swab configured to be inserted into an opening of the first chamber and to seal the first chamber with a slidable seal. The sample swab may be comprised of: a swab element, and a handle comprised of a seal portion configured to slide along the inner surface of the first chamber to slidably seal an internal cavity of the first chamber. The seal portion of the handle of the sample swab may be configured to slide along the liner to slidably seal the internal cavity of the first chamber when the swab element is inserted into the first chamber. In some embodiments, the sample swab is configured to be moved in a first direction to insert the swab element of the sample swab into the first chamber, and, when the sample swab is in an inserted position in the first chamber, the sample swab may be configured to be moved in a second direction, different from the first direction, to break one of or both of the first and second seals.

### BRIEF DESCRIPTION OF THE DRAWINGS

A skilled artisan will understand that the accompanying drawings are for illustration purposes only. It is to be understood that in some instances various aspects of the present technology may be shown exaggerated or enlarged to facilitate an understanding of the invention. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the present teachings in any way.

FIG. 1A depicts a perspective view of a test kit comprised of a diagnostic device and a crush-capable sample swab, according to some embodiments of the present technology.

FIG. 1B depicts a sectional view of the diagnostic device and the sample swab of FIG. 1A, according to some embodiments of the present technology.

FIG. 1C depicts a sectional view of the diagnostic device of FIG. 1A at a section through line 1C-1C in FIG. 1B, according to some embodiments of the present technology.

FIGS. 2A and 2B depict section views of the diagnostic device and the sample swab of FIG. 1A at various stages of a test procedure, according to some embodiments of the present technology.

FIG. 3A shows a flow chart for a method of using a diagnostic device and a sample swab, according to some embodiments of the present technology.

FIG. 3B shows a flow chart for a method of using a diagnostic device and a sample swab, according to some embodiments of the present technology.

FIGS. 4A through 4G graphically depict how a test kit comprised of a diagnostic device and a crush-capable sample swab may be used, according to some embodiments of the present technology.

FIG. 5 shows a flow chart for a method of making a diagnostic device, according to some embodiments of the present technology.

FIG. 6 shows a flow chart for a method of making a test kit, according to some embodiments of the present technology.

FIG. 7 depicts a perspective view of a test kit comprised of a diagnostic device and a crush-capable sample swab, according to some embodiments of the present technology.

FIG. 8A depicts a sectional views of the diagnostic device and the sample swab of FIG. 7, according to some embodiments of the present technology.

FIG. 8B depicts a sectional view of the diagnostic device of FIG. 7 at a section through line 8B-8B in FIG. 8A, according to some embodiments of the present technology.

FIGS. 8C through 8E depict sectional views of the diagnostic device and the sample swab of FIG. 7 at various stages of a test procedure, according to some embodiments of the present technology.

FIGS. 9A and 9B depict crush-capable sample swabs, according to some embodiments of the present technology.

### DETAILED DESCRIPTION

### 1. Introduction

The present disclosure provides test apparatuses, test kits, and methods of using the test apparatuses and/or the test kits (collectively referred to as “diagnostic systems” herein) for performing, in a clinical environment (e.g., medical facility, laboratory, etc.) and/or in a non-clinical environment (e.g., a home, a business office, a school, etc.), rapid diagnostic testing to detect one or more target nucleic-acid sequences in order to determine whether a subject has one or more diseases or ailments associated with the target nucleic-acid sequence(s). The diagnostic systems described herein, according to some embodiments of the present technology, may be self-administrable by a lay person and may be comprised of any combination of: a sample-collecting device (e.g., a swab), reagents, a diagnostic device that enables a reaction between the reagents and a sample, and a detection component, which may be included as part of the diagnostic device. As noted above, through use of, e.g., isothermal amplification methods, the apparatuses and techniques described herein may provide a diagnosis having an accuracy on par with the accuracy of typical PCR tests in less than one hour.

According to some embodiments of the present technology, the sample-collecting device may be a disposable swab configured to contact a test subject to collect the sample and to transfer the collected sample to the diagnostic device, and then may be discarded. In some other embodiments, the sample-collecting device may comprise part of the diagnostic device and may participate in a procedure of the test. For example, the sample-collecting component may facilitate an interaction between the sample and one or more of the reagents.

According to some embodiments of the present technology, the detection component may be an assay vehicle (e.g., a strip) on which is contained or attached one or more reagents for detecting the presence of a target nucleic-acid sequence indicative of a particular pathogen or disease. In some embodiments, the assay vehicle may contain or have attached thereto a plurality of reagents for detecting the presence of a plurality of different target nucleic-acid sequences indicative of a plurality of different pathogens or diseases. In some embodiments, the assay vehicle may be a lateral-flow assay (LFA) strip configured to come into contact with a sample solution and to enable the sample solution to flow through the strip from one end to another. Observable changes in a region of the LFA strip may indicate the presence of the target nucleic-acid sequence, indicating that the test subject may be afflicted with the pathogen or disease corresponding to the target nucleic-acid sequence. In some instances, for LFA strips that are able to detect more than one pathogen or disease, observable changes in multiple regions of the LFA strip may indicate the presence of multiple target nucleic-acid sequences, indicating that the test subject may be afflicted with more than one pathogen or disease corresponding to the target nucleic-acid sequences. In some embodiments, the detection component may be incorporated in the diagnostic device to, for example, minimize handling by a user, who may be a person without medical training. For example, the diagnostic device may be comprised of a window that may enable changes in an assay vehicle to be visible, which may enable a user to perform a reading of a test result and/or may enable an image (e.g., a photograph) of the assay vehicle to be captured and automatically read by a computer algorithm.

According to some embodiments of the present technology, the reagents may be comprised of any one or any combination of one or more lysis reagents, one or more nucleic-acid amplification reagents, one or more CRISPR/Cas detection reagents. The reagents may be in solid form (e.g., lyophilized, crystallized, etc.) and therefore, in some embodiments, included with the reagents may be one or more buffer solutions configured to activate one or more of the reagents. Additionally, included with the reagents may be one or more diluent fluids for achieving a desirable concentration of reagent fluids during various procedures of the test.

According to some embodiments of the present technology, a diagnostic device may comprise components for handling the reagents prior to their use in the test, components for storing and/or handling the reagents or the sample, or mixtures thereof, during various procedures of the test, and components for promoting reactions between the sample and one or more of the reagents. For example, such components may include one or more vessels (e.g., ampoules, capsules, vials, etc.) holding one or more reagents and/or one or more reaction fluids. In some embodiments, such vessels may be configured to be breakable (e.g., rupturable, crushable, burstable, etc.) to enable the one or more reagents and/or the one or more reaction fluids to mix with each other and/or with the sample during various procedures of the test. In some embodiments, such components may include one or more chambers or compartments holding one or more reagents, and/or one or more reaction fluids, and/or a LFA strip. In some embodiments, such chambers or compartments may be separated from each other by one or more seals.

According to some embodiments of the technology described herein, the diagnostic device may comprise one or more components configured to move with one or more movements of a sample swab inserted in a sample chamber of the diagnostic device during a test procedure. The sample swab may be configured to seal the sample chamber upon insertion, and to provide a movable seal such that sliding the sample swab in different directions (e.g., inwards and outwards) relative to a base of the sample chamber may change an internal volume of the sample chamber while maintaining a fluid-tight seal of the sample chamber. Movement of the sample swab may cause a burstable capsule to burst or be ruptured directly or indirectly by a portion of the sample swab, to enable contents of the capsule to be released to interact with a sample carried on a swab element of the sample swab.

In some embodiments of the present technology, the sample chamber may be separated from fluid communication with one or more other chamber(s) by one or more breakable seal(s). The breakable seal(s) may break via movement of the sample swab relative to a housing containing the sample chamber. At should be understood that “breaking” a breakable seal may not involve tearing or ripping of a structure but may instead involve movement of the breakable seal from a position blocking, e.g., a conduit to a position in which the conduit is not blocked by the breakable seal. In some embodiments, the sample chamber may be comprised of a movable liner forming a part of an inner surface of an internal cavity of the sample chamber. The sample swab may be comprised of a seal portion that comes into contact with the liner and seals the sample chamber when the sample swab is inserted into the sample chamber (e.g., when the swab element of the sample swab is inserted in a first direction toward the base of the sample chamber). The seal formed between the liner and the seal portion of the sample swab may be a fluid-tight seal. During insertion, the seal portion of the sample swab may glide or slide relative to the liner. For example, during insertion, a base end of the liner may abut a ledge or a surface of the base of the sample chamber when the seal portion of the sample swab slides in the first direction into the sample chamber. The liner may not move appreciably relative to the base of the sample chamber when the sample swab is inserted in the first direction toward the base of the sample chamber. In such a case, the seal portion of the sample swab may form a movable seal with the liner, in which a location of the seal on the liner may move when the sample swab moves in the first direction.

In some embodiments of the present technology, movement of the sample swab in the first direction during insertion of the sample swab into the sample chamber may cause a portion of the sample swab to contact and rupture a burstable capsule containing a fluid. For instance, the capsule may be located in the sample chamber and may be ruptured by the swab element or another portion of the sample swab during insertion of the sample swab into the sample chamber. A handle of the sample swab may have visible markings indicating an insertion depth of the sample swab into the sample chamber, so a user may have a visible indication of whether the sample swab has been inserted to a desirable depth for each step of the test procedure. In some embodiments, the capsule may be spaced apart from the surface of the base of the chamber by a spacer object (e.g., a ring, a ledge, etc.), such that the swab element of the sample swab may pierce through the capsule into a pocket of the sample chamber between the capsule (pre-piercing) and the base of the sample chamber. Once ruptured (e.g., by piercing, crushing, squeezing, etc.), fluid in the capsule my flow into the sample chamber and may interact with the sample carried on the swab element of the sample swab inserted in the sample chamber. In some embodiments, the capsule may sit directly on the surface of the base of the sample chamber. As will be appreciated, the sample swab may have a length sufficient to reach the capsule and rupture the capsule during insertion.

In some embodiments of the present technology, movement of the sample swab in the first direction during insertion of the sample swab into the sample chamber may cause a contact portion of the sample swab to contact and move a movable crusher in the housing, to cause the crusher to rupture the capsule. The sample swab may have a protrusion (e.g., a protruding edge, a protruding tab, etc.) configured to contact the crusher directly or indirectly to push the crusher against the capsule to crush and rupture the capsule. Thus, the sample swab may indirectly cause the capsule to rupture via the crusher. For example, a force exerted by the user to insert the sample swab into the sample chamber and to push the sample swab to an inserted position may be at least partially transferred by the protrusion to the crusher as a rupture force, to urge the crusher against the capsule to rupture the capsule. Alternative ways to rupture the capsule directly or indirectly by a sample swab are envisioned, as well as ways of rupturing the capsule that may not involve a sample swab.

In some embodiments of the present technology, the capsule may contain a reagent fluid (e.g., an amplification fluid), which may interact with the sample carried on the swab element of the sample swab to form a sample fluid in the sample chamber. In some other embodiments, the capsule may contain a buffer fluid configured to activate a lyophilized reagent (e.g., an amplification tablet) located in the sample chamber. A sample fluid may form from an interaction of the buffer fluid, the reagent, and the sample in the sample chamber.

As noted above, the sample chamber may be comprised of a movable liner forming a part of the inner surface of the internal cavity of the sample chamber. In some embodiments of the present technology, after the sample fluid has formed in the sample chamber through insertion of the sample swab to an insertion position (e.g., a position at which the capsule is ruptured), the sample swab may be moved (e.g., pulled) in a second direction different from the first direction in which the sample swab is inserted into the sample chamber. In some embodiments, the second direction may be opposite to the first direction. Such movement in the second direction may cause the seal portion of the sample swab to move together with the liner in the second direction. For example, if the first direction is an insertion direction, the second direction may be an opposite direction in which the sample swab is pulled outward and away from the base of the sample chamber. The corresponding movement of the liner with the sample swab in the second direction may cause the breakable seal(s) separating the sample chamber from the other chamber(s) to break, thus enabling the sample chamber to be in fluid communication with the other chamber(s). In some embodiments, the breakable seal(s) may be attached to the liner and may physically break (e.g., rip) when the liner moves in the second direction. In some embodiments, the breakable seal(s) may be cover(s) that cover and seal opening(s) to the sample chamber. In such cases, movement of the liner may uncover or unseal the opening(s) to the sample chamber without physical breaking of material forming the seal(s).

In some embodiments of the present technology, the other chamber(s) may comprise one or both of: a diluent chamber containing a diluent fluid and a test chamber containing a test and readout vehicle. In various embodiments described herein a linear-flow assay (LFA) strip is used as the test and readout vehicle. However, it should be understood that other test and readout vehicles are envisioned, and the technology presented herein is not limited to use with test and read-out vehicles that are LFA strips. When a breakable first seal separating the sample chamber and the diluent chamber breaks, the diluent fluid may combine with the sample fluid in the sample chamber to form a diluted sample fluid. Similarly, when a breakable second seal separating the sample chamber and the test chamber breaks, the diluted sample fluid may be allowed to reach the LFA strip and to reach (e.g., via immersion or via capillary action) test portions of the LFA strip, which may be configured to detect one or more target nucleic acid sequence(s) corresponding to one or more pathogen(s). In some embodiments, the test chamber may have a window through which the LFA strip is visible, to enable the LFA strip to be read by a human and/or by a machine.

In some embodiments of the present technology, breakage of the first and second seals may occur simultaneous or sequentially. For example, the sample swab may be moved from the inserted position in the second direction to a breakage position, which may simultaneously (or nearly simultaneously) break the first and second seals separating the sample chamber from the diluent chamber and from the test chamber. In another example, the sample swab may be moved from the inserted position in the second direction to a first breakage position, which may break the first seal separating the sample chamber from the diluent chamber. Subsequently, the sample swab may be moved further in the second direction, from the first breakage position to a second breakage position, which may break the second seal separating the sample chamber from the test chamber. As noted above, the handle of the sample swab may have visible markings indicating any one or any combination of: the insertion position, the first breakage position, and the second breakage position, thus avoiding user confusion as to where the sample swab should be located for various steps or phases of the test procedure.

In some embodiments of the present technology, a heater may be provided to heat the sample chamber (e.g., to heat the sample fluid and/or the reagent fluid).

2. Test Systems and Components with Movable Seals

2.1 Diagnostic Device with Chamber Sealable by Movable Plug Seal

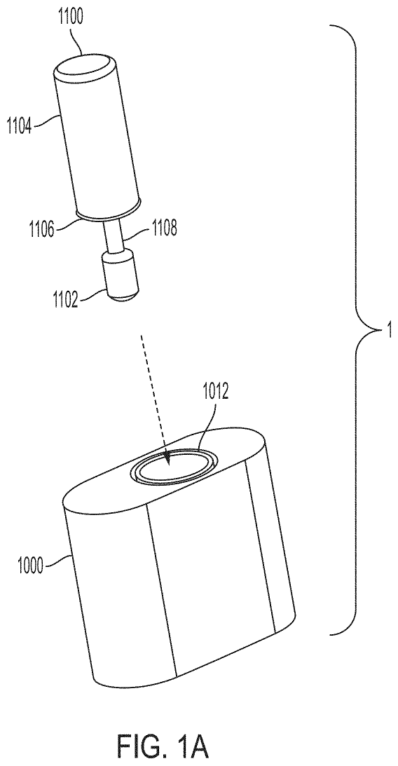

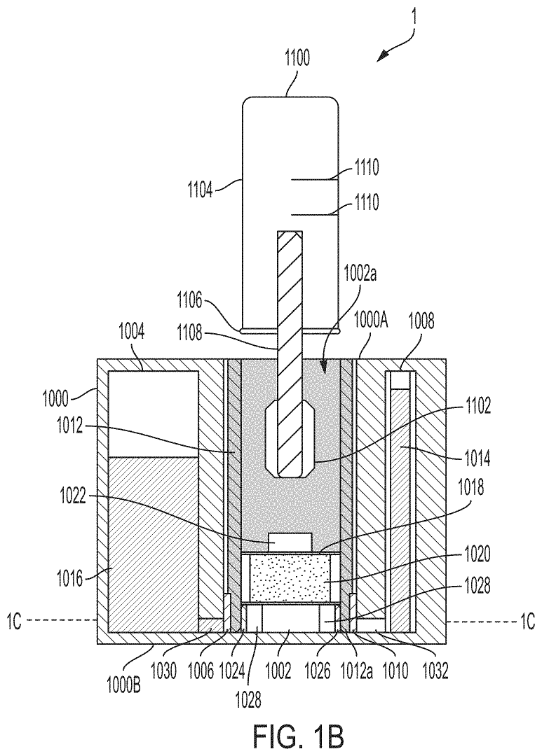

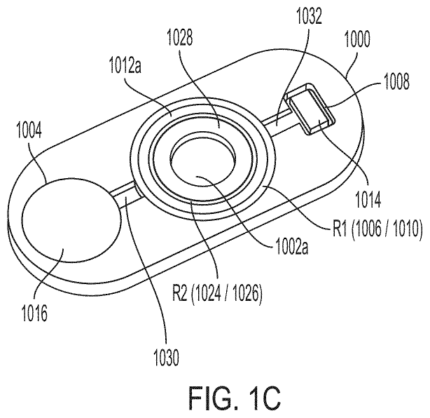

FIG. 1A schematically depicts a perspective view of a diagnostic device 1000 and a sample swab 1100 configured to form a plug seal with the diagnostic device 1000 during a rapid diagnostic test procedure, according to some embodiments of the present technology. The seal need not be at a fixed position between the sample swab 1100 and the diagnostic device 1000, but may be a movable seal. That is, the seal between the sample swab 1100 and the diagnostic device 1000 may not be fixed at a single position but instead may be a slidable seal having a position that varies depending on an insertion position of the sample swab 1100 relative to the diagnostic device 1000. The diagnostic device 1000 and the sample swab 1100 may be included as part of a test kit 1.

FIG. 1B schematically depicts a sectional view of the diagnostic device 1000 and the sample swab 1100, according to some embodiments of the present technology. The diagnostic device 1000 may be comprised of a first chamber 1002, a second chamber 1004 separated from the first chamber 1002 by a breakable first seal 1006, and a third chamber 1008 separated from the first chamber 1002 by a breakable second seal 1010. The diagnostic device 1000 also may be comprised of a movable liner 1012 forming a portion of an inner surface of the first chamber 1002.

According to some embodiments of the present technology, the sample swab 1100 may be configured to be inserted into an internal cavity 1002a of the first chamber 1002 via an opening of the first chamber 1002. The sample swab 1100 may plug the opening of the first chamber 1002 and slidably seal the first chamber 1002. The sample swab 1100 may be comprised of a swab element 1102 attached to a handle 1104 via a stem 1108. The handle 1104 may be comprised of a seal portion 1106 configured to contact the liner 1012 to seal the first chamber 1002. The seal portion 1106 may slide along the liner 1012 while the sample swab 1100 is being moved to an inserted position in the first chamber 1002. In FIG. 1B, the sample swab 1100 is depicted to be partially inserted into the first chamber 1002 but the first chamber 1002 is not yet sealed by the sample swab 1100.

In use, as the swab element 1102 of the sample swab 1100 may be inserted into the first chamber 1002, the seal portion 1106 of the sample swab 1100 may press against the liner 1012 to plug the first chamber 1002 and provide a fluid-tight seal that moves as the swab element 1102 is moved toward a base of the first chamber 1002, according to some embodiments of the present technology. In some embodiments, the seal portion 1106 may be comprised of a resilient gasket (e.g., an o-ring) configured to undergo compression during insertion, such that the gasket is pressed against the liner 1012. In some embodiments, the seal portion 1106 may be formed of a polymeric material that enables the seal portion 1106 to slide along the liner 1012 when a user pushes on the handle 1104 to insert the sample swab 1100. As will be appreciated, a sealed volume of the internal cavity 1002a of the first chamber 1002 may vary according to an insertion depth or location of the seal portion 1106 of the sample swab 1100 relative to the base of the first chamber 1002.

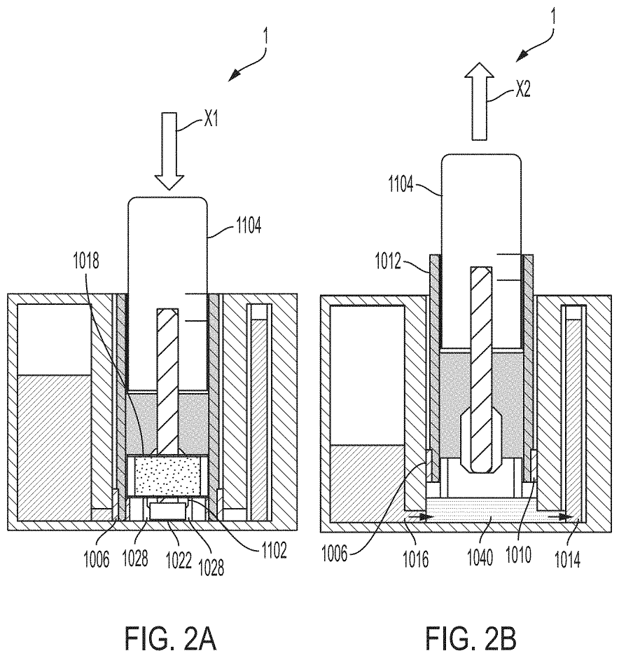

According to some embodiments of the present technology, the handle 1104 of the sample swab 1100 may be comprised of visible markings 1110. A user may use a location of the markings 1110 relative to an edge 1000A or other surface of the diagnostic device 1000 to determine an insertion depth of the sample swab in the first chamber 1002. Optionally, the markings may be color coded to indicate different levels of depth. In some embodiments, schematically depicted in FIGS. 2A and 2B, the sample swab 1100 may be configured to be moved in a first direction 11 to insert the swab element 1102 of the sample swab into the first chamber 1002 to the inserted position (or another position), which may align one of the markings 1110 with the edge 1000A of the apparatus 1000. The sample swab 1100 also may be configured to be moved in a second direction 12, different from the first direction 11, to break one or both of the first and second seals 1006, 1010, as discussed below. In some embodiments, the second direction X2 may be opposite to the first direction X1.

FIG. 1C schematically depicts a sectional view of the diagnostic device 1000 in a plane through a line identified by 1C-1C in FIG. 1B, near a base end 1000B of the diagnostic device 1000, according to some embodiments of the present technology. In some embodiments, a base end 1012a of the liner 1012 may be sandwiched between an outer ring R1 and an inner ring R2. The outer ring R1 may be configured to cover and seal a first opening or conduit 1030 between the first chamber 1002 and the second chamber 1004, and may form at least a part of the first seal 1006 separating the first chamber 1002 from the second chamber 1004. The outer ring R1 also may be configured to cover and seal a second opening or conduit 1032 between the first chamber 1002 and the third chamber 1008, and may form part of the second seal 1010 separating the first chamber 1002 from the third chamber 1008. In some embodiments, when the sample swab 1100 is moved in the second direction X2, the seal portion 1106 of the sample swab may pull the liner 1012 in the second direction X2, thus causing the outer ring R1 to be pulled in the second direction X2 away from the base end 1000B of the diagnostic device 1000. Movement of the outer ring R1 away from the base end 1000B of the diagnostic device 1000 may cause the first seal 1006 to break (e.g., to move away from a sealing position), to enable the first and second chambers 1002, 1004 to be in fluid communication with each other via the first opening or conduit 1030. Similarly, movement of the outer ring R1 away from the base end 1000B of the diagnostic device 1000 may cause the second seal 1010 to break, to enable the first and third chambers 1002, 1008 to be in fluid communication with each other via the second opening or conduit 1032. As noted above, breakage of the first seal 1006 and/or the second seal 1010 may comprise moving the outer ring R1 from covering the first opening or conduit 1030 and/or the second opening or conduit 1032, and need not involve ripping or tearing of material.

According to some embodiments of the present technology, a distance or height of the first opening or conduit 1030 relative to the base end 1000B may be the same as a distance or height of the second opening or conduit 1032 relative to the base end 1000B, in which case the first and second seals 1006, 1010 may break simultaneously or nearly simultaneously when the outer ring R1 is pulled in the second direction X2 through movement of the sample swab 1100 in the second direction X2. In some embodiments, the height of the first opening or conduit 1030 relative to the base end 1000B may be the different from the height of the second opening or conduit 1032 relative to the base end 1000B, in which case the first and second seals 1006, 1010 may break sequentially, with the order of breakage depending on which opening or conduit is closer to the base end 1000B. For example, if a position of the first opening or conduit 1030 is relatively closer to the base end 1000B compared to a position of the second opening or conduit 1032 relative to the base end 1000B, the first seal 1006 may break first when the outer ring R1 is pulled in the second direction X2 from an initial position at which the liner 1012 is fully inserted in the first chamber 1002. Subsequently, further pulling of the outer ring R1 in the second direction X2 may cause the second seal 1010 to break. The markings 1110 on the handle 1104 may guide the user to pull to a first pull position at which the first seal 1006 may break, and to pull to a second pull position at which the second seal 1010 may break. As will be appreciated, a height of the outer ring R1 may be sufficiently tall to cover simultaneously the first opening or conduit 1030 and as well as the second opening or conduit 1032, in some embodiments.

In some embodiments of the present technology, the inner ring R2 may aid in positioning the base end 1012a of the liner 1012 relative to an annular spacer 1028 defining a base portion of the first chamber 1002. The inner ring R2 may be comprised of a first portion 1024 sandwiching the base end 1012a of the liner 1012 with the first seal 1006 at the first opening or conduit 1030, and a second portion 1026 sandwiching the base end 1012a of the liner 1012 with the second seal 1010 at the second opening of conduit 1032.

In some embodiments of the present technology, the liner 1012 may be configured such that, when the liner 1012 is fully inserted in the first chamber 1002, an outer edge of the liner 1012 may align with the edge 1000A of the diagnostic device 1000, as schematically depicted in FIG. 1B. When fully inserted, the liner 1012 may be at a stop position at which the base end 1012a of the liner 1012 may abut a ledge or a surface of the first chamber 1002. Alternatively, in some embodiments, the inner ring R2 may have a ledge on which the liner 1012 may sit when the liner 1012 is fully inserted in the first chamber 1002. As will be appreciated, when the sample swab 1100 is inserted in the first chamber 1002, the seal portion 1106 of the sample swab 1100 may slide in the first direction X1 relative to the liner 1012 because the liner 1012 may be at the stop position and therefore may not be moved further in the first direction X1. On the other hand, when the sample swab 1100 is moved in the second direction X2, the seal portion 1106 may pull the liner 1012 in the second direction X2 such that the seal portion 1106 and the liner 1012 may move together in the second direction X2.

As will be appreciated, although the first and second seals 1006, 1010 are shown in FIG. 1C to be part of the outer ring R1, in some embodiments of the present technology the first and second seals 1006, 1010 may be configured differently and may not be connected to a common structure. Similarly, instead of the first and second portions 1024, 1026 being connected together via the inner ring R2, the first and second portions 1024, 1026 may be configured differently and may, e.g., be discrete structures that are separate from each other.

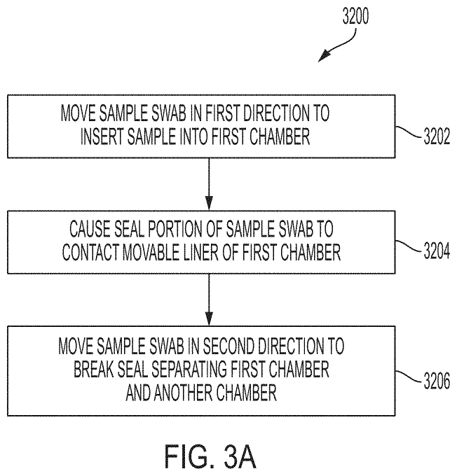

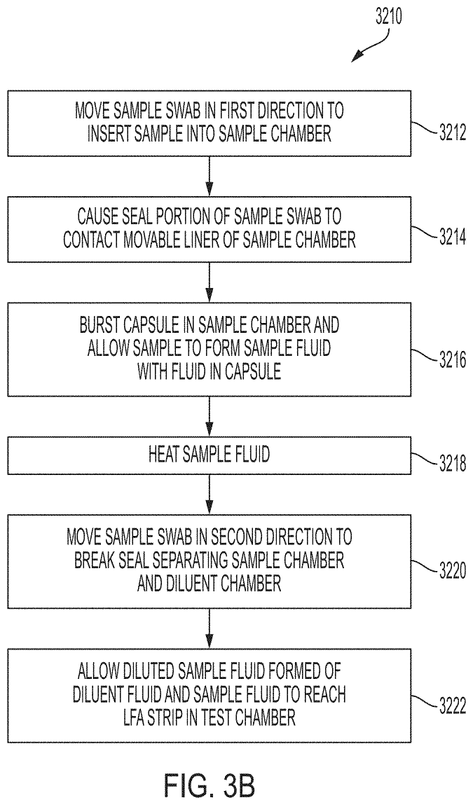

FIG. 3A shows a flow chart summarizing a method 3200 of using the diagnostic device 1000 and the sample swab 1100, according to some embodiments of the present technology. The method 3200 may be understood with reference to FIGS. 1B, 2A, and 2B. According to the method 3200, at act 3202, the sample swab 1100 may be moved in the first direction X1 by a user to insert the sample swab 1100 into the first chamber 1002. For example, the swab element 1102 of the sample swab 1100 may carry a sample obtained from, e.g., a nasal cavity or an oral cavity of a subject. At act 3204, the seal portion 1106 of the sample swab 1100 may make contact with the liner 1012 as the sample swab 1100 is moved in the first direction X1 to an inserted position indicated by, e.g., a marking 1110 on the handle 1104 of the sample swab 1100. For instance, FIG. 2A depicts the sample swab 1100 in a fully inserted position in the first chamber 1002. As the sample swab 1100 is inserted to the fully inserted position, the seal portion 1106 may slide or glide in the first direction X1 relative to the liner 1012. At act 3206, the user may move the sample swab 1100 in the second direction X2 away from the fully inserted position. Movement of the seal portion 1106 of the sample swab 1100 in the second direction X2 may pull the liner 1012 such that the seal portion 1106 and the liner 1012 may move together in the second direction X2. For example, a friction force between the liner 1012 and the seal portion 1106 may be sufficient for the liner 1012 to be pulled away from the stop position when the sample swab 1100 is pulled away from the fully inserted position. When the liner 1012 is moved a sufficient distance (e.g., 0.5 inch or 1 inch, or 1.5 inches, or 2 inches, or 2.5 inches), one or both of the first and second seals 1006, 1010 may break.

In an example implementation of the present technology, the first chamber 1002 may be a sample chamber 1002 and may contain a reagent 1022 and a burstable capsule 1018 containing a first fluid 1020. The reagent 1022 may be, e.g., a lyophilized amplification reagent and the first fluid 1020 may be a buffer fluid configured to activate the lyophilized amplification reagent. In some embodiments of this implementation, the capsule 1018 may rest on the annular spacer 1028 such that a pocket or space is present between the capsule 1018 and a base surface of the sample chamber 1002, as depicted in FIG. 1B. In an alternative embodiment of this implementation, the sample chamber 1002 may not contain a reagent but instead the capsule 1018 may contain a reagent fluid (e.g., an amplification fluid). In some embodiments of this implementation, the second chamber 1004 may be a diluent chamber containing a second fluid 1016, which may be diluent fluid. In some embodiments of this implementation, the third chamber 1008 may be a test chamber and may contain a lateral-flow assay (LFA) strip 1014 configured to detect a presence of one or more pathogen(s) in a sample obtained from a subject to be tested. The first seal 1006 may separate the second (diluent) fluid 1016 from the reagent 1022 and the capsule 1018 containing the first (buffer) fluid 1020 in the sample chamber 1002, and the second seal 1010 may separate the LFA strip 1014 in the test chamber 1008 from contents of the sample chamber 1002.

FIG. 3B shows a flow chart for a method 3210 of using the example implementation of the diagnostic device 1000 and the sample swab 1100 described in the previous paragraph, according to some embodiments of the present technology. The method 3210 may be understood with reference to FIGS. 1B, 2A, and 2B. According to the method 3210, at act 3212, the sample swab 1100 may be moved in the first direction X1 by a user to insert the sample swab 1100 into the sample chamber 1002. For example, the swab element 1102 of the sample swab 1100 may carry a sample obtained from, e.g., a nasal cavity or an oral cavity of a subject. At act 3214, the seal portion 1106 of the sample swab 1100 may contact the liner 1012 as the sample swab 1100 is moved in the first direction X1. As the sample swab 1100 is inserted to a fully inserted position, the seal portion 1106 may slide or glide in the first direction X1 relative to the liner 1012 and thus may movably seal the sample chamber 1002. At act 3216, the sample swab 1100 may reach the fully inserted position, which may be indicated by, e.g., a marking 1110 on the handle 1104 of the sample swab 1100. For instance, as depicted in FIG. 2A, in reaching the fully inserted position a portion of the sample swab 2100 (e.g., the swab element 1012 or another portion) may push the reagent 1022 through the capsule 1018 into the space between the capsule 1018 and the base surface of the sample chamber 1002 (i.e., the space delineated by the annular spacer 1028), thus rupturing the capsule 1018 and releasing the first (buffer) fluid 1016 into the sample chamber 1002. In some embodiments, interaction of the reagent 1022, the first (buffer) fluid 1016, and the sample carried by the swab element 1102 of the sample swab 1100 may form a sample fluid in the sample chamber 1002. At act 3218, the sample fluid may be heated by a heater (not shown). At act 3220, the user may move the sample swab 1100 in the second direction X2 away from the fully inserted position. Movement of the seal portion 1106 of the sample swab 1100 in the second direction X2 may pull the liner 1012 away from the stop position, such that the seal portion 1106 and the liner 1012 may move together in the second direction X2. When the liner 1012 is moved a sufficient distance (e.g., 0.5 inch or 1 inch, or 1.5 inches, or 2 inches, or 2.5 inches) in the second direction X2, one or both of the first and second seals 1006, 1010 may break. At act 3220, after the first seal 1006 breaks, the second (diluent) fluid 1016 in the second chamber 1004 may flow into the sample chamber 1002 and may interact with the sample fluid. At act 3222, after the second seal 1010 breaks, a diluted sample fluid 1040 formed from the second (diluent) fluid 1016 and the sample fluid may flow into the test chamber 1008 and may contact the LFA strip 1014.

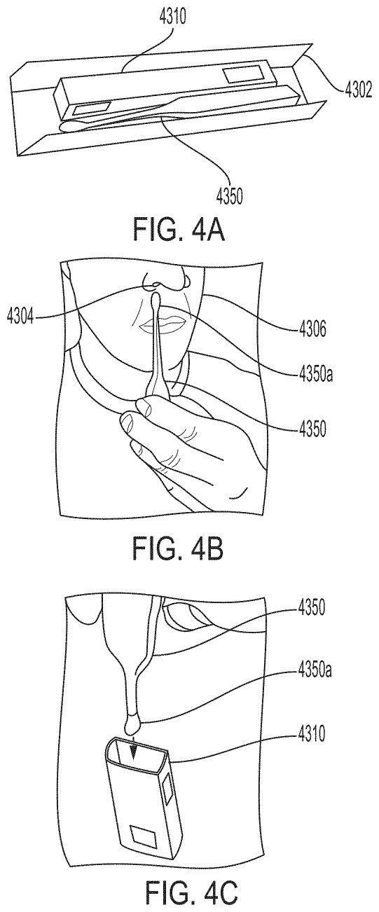

FIGS. 4A through 4G graphically illustrate how a test kit comprised of a diagnostic device 4310 (e.g., the diagnostic device 1000) and a sample swab 4350 (e.g., the sample swab 1100) may be used in a test procedure, according to some embodiments of the present technology. FIG. 4A depicts the diagnostic device 4310 and the sample swab 4350 taken out of packaging material 4302 (e.g., wrapper(s), box(es), etc.). FIG. 4B depicts a subject 4306 about to have a sample taken from his/her nasal cavity 4304. The subject 4306 or a user (e.g., a nurse, a family member, an assistant) may obtain the sample by swabbing or contacting a surface of the subject's nasal cavity 4304 with a swab element 4350a of the sample swab 4350. FIG. 4C depicts the swab element 4350a of the sample swab 4350 about to be inserted into the diagnostic device 4310. For example, the sample swab 4350 may be inserted into an opening of a sample chamber of the diagnostic device 4310. FIG. 4D depicts a test assembly 4300 comprised of the sample swab 4350 inserted into the diagnostic device 4310 during the test procedure. For example, the sample swab 4350 may be comprised of a seal portion (e.g., the seal portion 1106) that may form a leak-tight seal with a movable liner (e.g., the liner 1012) forming at least a portion of an internal cavity of the sample chamber of the diagnostic device 4310. The diagnostic device 4310 may be comprised of a reaction window 4312 at a base end of the diagnostic device 4310, to enable the subject 4306 or the user to ascertain whether the swab element 4350a has been properly inserted into a fully inserted position. In some embodiments, the window 4312 may enable the subject 4306 or the user to see whether the swab element 4350a is interacting with a fluid, indicating that a burstable capsule (e.g., the capsule 1018) containing the fluid has been ruptured. In some embodiments, the capsule and a reagent may be in the sample chamber and may undergo a reaction with the sample to form a sample fluid. The diagnostic device 4310 also may be comprised of a detection window 4320, discussed below.

FIG. 4E depicts the test assembly 4300 about to be inserted into a recess 4360a of a heater 4360. The recess 4360a may be configured to fit only the base end of the diagnostic device 4310, to ensure that the sample fluid in the sample chamber undergoes heating, and to prevent a handle of the sample swab 4350 from erroneously being heated. FIG. 4F depicts the test assembly 4300 being removed from the heater 4360. After the heating has completed, the sample swab 4350 may be pulled away from the base end of the diagnostic device 4310, which may cause the liner to be pulled away and which consequently may cause a seal separating the sample chamber from diluent fluid in a diluent chamber to be broken. The diluent fluid and the sample fluid may combine to form a diluted sample fluid. When the liner is pulled away by the sample swab 4350, a seal separating the sample chamber from an LFA strip in a test chamber may be broken, which may enable the diluted sample fluid to reach the LFA strip, as described above. Optionally, the pulling of the liner and the sample swab 4350 away from the base end of the diagnostic device 4310 may occur after heating while the test assembly 4300 is still in the recess 4360a of the heater 4360. FIG. 4G depicts test regions of the LFA strip visible through the detection window 4320. After the diluted sample fluid has been allowed to interact with the test regions of the LFA strip, a change in an appearance of the test regions may occur and may be seen through the detection window 4320.

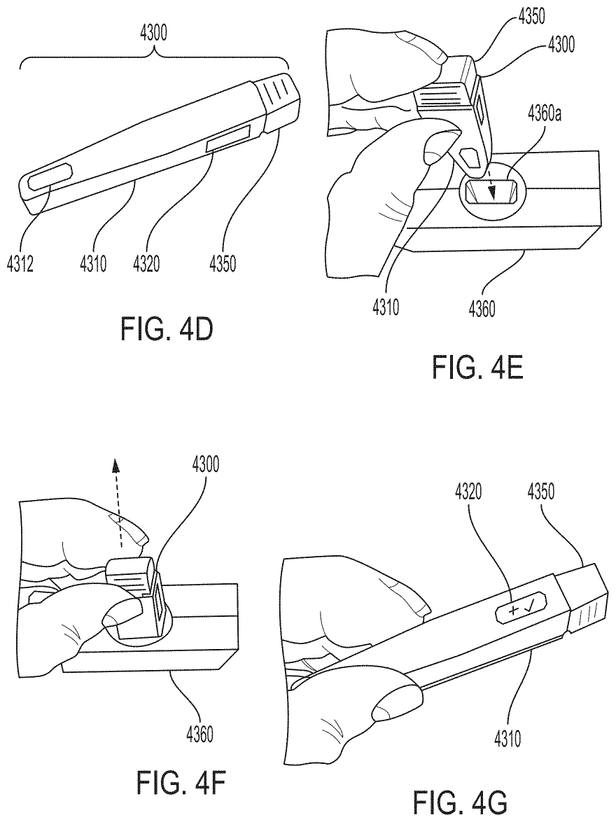

FIG. 5 shows a flow chart summarizing a method 5000 for manufacturing an diagnostic device (e.g., the diagnostic device 1000) usable with a sample swab (e.g., the sample swab 1100) that forms a movable plug seal with the diagnostic device, according to some embodiments of the present technology. According to the method 5000, at act 5002, a movable liner (e.g., the liner 1012) may be inserted in a first chamber (e.g., the sample chamber 1002) of the diagnostic device. At act 5004, a fluid (e.g., the second fluid 1016) may be added to a second chamber (e.g., the second chamber 1004) of the diagnostic device. A breakable seal (e.g., the first seal 1006) may be present between the first and second chambers or may be added prior to adding the fluid to the second chamber. For example, a polymeric ring-type seal structure (e.g., the outer ring R1) may be inserted in the first chamber. At act 5006, a test vehicle (e.g., the LFA strip 1014) may be added to a third chamber (e.g., the third chamber 1008) of the diagnostic device. At act 5008, a burstable capsule (e.g., the capsule 1018) may be added to the first chamber. The liner may be configured to form a seal with a sample swab (e.g., with the seal portion 1106 of the sample swab 1100), such that the sample swab may plug an opening of the first chamber. The seal may be a movable seal, such that sample swab may seal the opening of the first chamber as the sample swab is moved in a first direction to an inserted position in the first chamber. The liner also may be configured to move with the sample swab as the sample swab is pulled in a second direction different from the first direction, i.e., outwards relative to the first chamber and away from the inserted position. Movement of the liner outwards from the first chamber may cause the seals separating the first chamber from the second and third chambers to break.

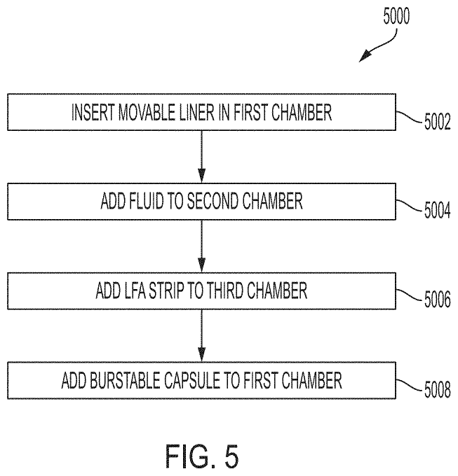

FIG. 6 shows a flow chart summarizing a method 6000 for manufacturing a rapid diagnostic test kit that utilizes a sample swab that forms a movable plug seal with a sample chamber, according to some embodiments of the present technology. According to the method 6000, at act 6002, a diagnostic device (e.g., the diagnostic device 1000) may be provided. The diagnostic device may be comprised of a sample chamber and a movable liner forming an inner surface of the sample chamber. At act 6004, a sample swab (e.g., the sample swab 1100) may be provided. The sample swab may be configured to seal the sample chamber of the test apparatus when a portion of the sample swab contacts the liner. The seal formed by the sample swab may be a movable plug seal. At act 6006, the diagnostic device and the sample swab may be packaged together as a single unit. Optionally, a heater may be included in the test kit, and may be packaged with the test apparatus and the sample swab as the single unit.

2.2 Diagnostic Device with Crusher and Chamber Sealable by Movable Sample Swab

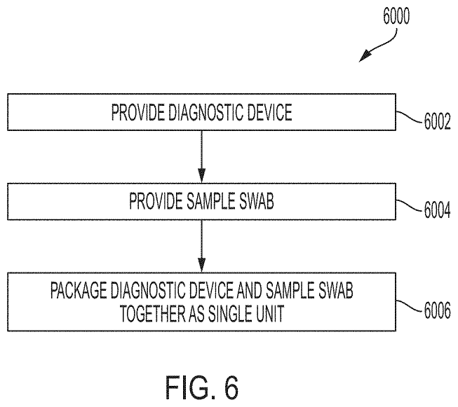

FIG. 7 schematically depicts a perspective view of a diagnostic device 7410 and a sample swab 7450 configured to form a plug seal with the diagnostic device 7410 during a rapid diagnostic test procedure, according to some embodiments of the present technology. The seal formed by the sample swab 7450 may be movable relative to the diagnostic device 7410. The diagnostic device 7410 and the sample swab 7450 may be included as part of a test kit 7400.

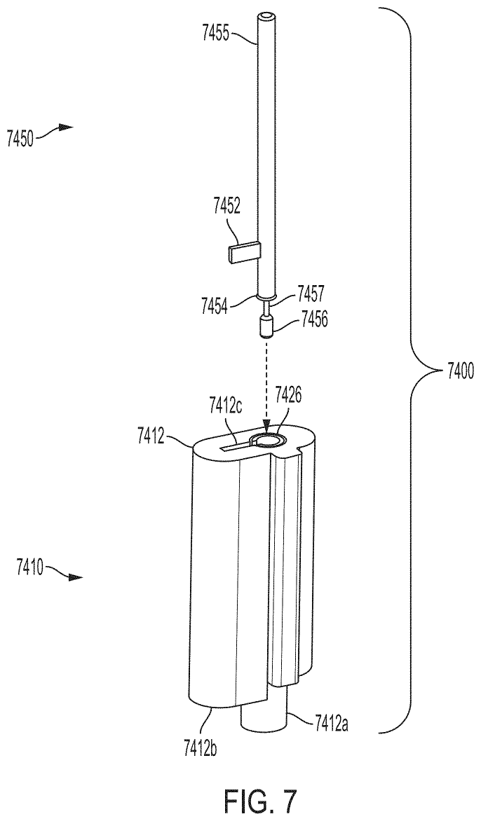

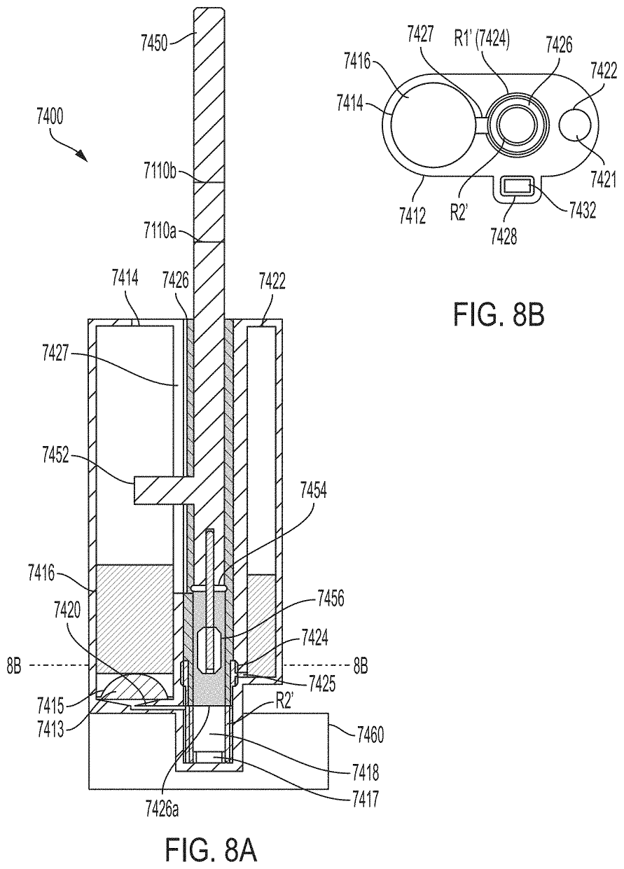

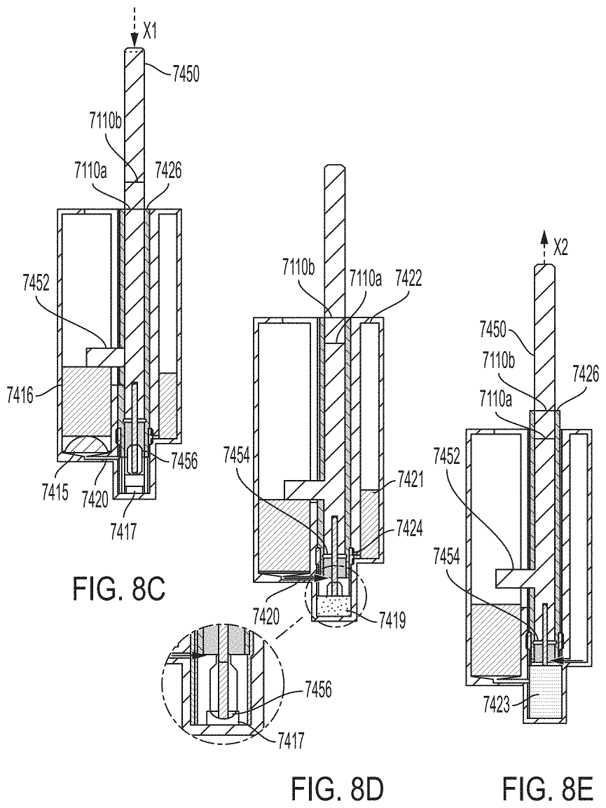

FIGS. 8A through 8E schematically depict sectional views of the diagnostic device 7410 and the sample swab 7450, according to some embodiments of the present technology. The diagnostic device 7410 may be comprised of a housing 7412, a first chamber 7414 disposed in the housing 7412, and a sample chamber 7418 disposed in the housing 7412. The first chamber 7414 may be comprised of a burstable capsule 7415 and a crusher 7416 configured to move in the first chamber 7414 to rupture the capsule 7415 to release a fluid 7413 contained in the capsule 7415. The sample chamber 7418 may be configured to receive the sample swab 7450 through an opening of the sample chamber 7418. The crusher 7416 may be configured to be moved in the first chamber 7414 by direct or indirect contact with a contact portion 7452 of the sample swab 7450 when the sample swab 7450 is inserted in the sample chamber 7418. In some embodiments, the contact portion 7452 of the sample swab 7450 may contact a surface of the crusher 7416 during insertion of a swab element 7456 of the sample swab 7450 into the sample chamber 7418. As the swab element 7456 and the contact portion 7452 of the sample swab 7450 move in a first direction X1 (see FIG. 8C) toward an inserted position in the sample chamber 7418, the contact portion 7452 may exert a force on the crusher 7416 to move the crusher 7416 in the first direction X1. The crusher 7416 may, in turn, contact and exert a crushing force on the capsule 7415 to cause the capsule 7415 to rupture. According to some embodiments, the capsule 7415 may be a pod comprised of a rupturable shell or skin in which the fluid 7413 is confined. In some embodiments, a rupture force for rupturing the capsule 7415 may have a value between 0.2 pound and 1.5 pounds, or between 1 pound and 2 pounds, or between 0.5 pound and 3 pounds.

FIG. 8A depicts an example of the crusher 7416 in a rest position, in which the crusher 7416 is not in contact with the contact portion 7452 of the sample swab 7450, according to some embodiments of the present technology. In the rest position, the crusher 7416 may be spaced apart from the capsule 7415. FIG. 8C depicts an example of the crusher 7416 in a standby position in some embodiments, in which the contact portion 7452 of the sample swab 7450 may be in contact with the crusher 7416. In the standby position, the crusher 7416 may be in contact with and the capsule 7415 but may not apply a crushing force on the capsule 7415. FIG. 8D depicts an example of the crusher 7416 in a crush position in some embodiments, in which movement of the contact portion 7452 of the sample swab 7450 has caused the crusher 7416 to move with sufficient force that the crusher 7416 has in turn exerted a force on the capsule 7415 meeting or exceed the crushing force. In FIG. 8D, the capsule 7415 has been ruptured.

In some embodiments of the present technology, a conduit 7420 may connect the sample chamber 7418 to the first chamber 7414, such that these chambers 7418, 7414 may be in fluid communication with each other. In some embodiments, when the crusher 7416 causes the capsule 7415 to rupture, the fluid 7413 in the capsule 7415 may flow from the first chamber 7414 to the sample chamber 7418 via the conduit 7420.

In some embodiments of the present technology, a reagent 7417 may be disposed in the sample chamber 7418, and the fluid 7413 in the capsule 7415 may be a buffer fluid configured to activate the reagent 7417. For example, the reagent 7417 may be a lyophilized amplification reagent configured to be activated when in contact with the (buffer) fluid 7413 from the capsule 7415. In some embodiments, when the sample swab 7450 is in the fully inserted position (e.g., shown in FIG. 8D), the reagent 7417, the (buffer) fluid 7413 from the capsule 7415, and a sample carried on the swab element 7456 of the sample swab 7450 may interact with each other in the sample chamber 7418 to form a sample fluid 7419.

In some embodiments of the present technology, the sample chamber 7418 may be heated by a heater 7460. The sample chamber 7418 may be arranged in the housing 7412 such that the sample chamber 7418 may be readily heated by the heater 7460. In some embodiments, the housing 7412 may be comprised of a protrusion 7412a extending from a base 7412b of the housing 7412. The sample chamber 7418 may be located in the protrusion 7412a, and the protrusion 7412a may be configured to be received in a recess of the heater 7460, as schematically depicted in FIG. 8A.

In some embodiments of the present technology, a movable liner 7426 may form a portion of an inner surface of the sample chamber 7418. A seal portion 7454 of the sample swab 7450 may come into contact and slide along the liner 7426 when the swab element 7456 of the sample swab 7450 is inserted into the sample chamber 7418. In some embodiments, when the sample swab 7450 is moved in the first direction X1 during insertion of the sample swab 7450 to the inserted position in the sample chamber 7418, the seal portion 7454 of the sample swab 7450 may slide in the first direction X1 relative to the liner 7426. A seal between the seal portion 7454 of the sample swab 7450 and the liner 7426 may move in the first direction X1 along with movement of the sample swab 7450, and thus may be considered a movable seal. In some embodiments, when the sample swab 7450 is moved in a second direction X2 different from to the first direction X1, the liner 7426 and the seal portion 7454 of the sample swab 7450 may slide together in the second direction X2, and thus the liner 7426 may be considered to be pulled by the sample swab 7450. In some embodiments, the second direction X2 may be opposite to the first direction X1, as depicted in FIGS. 8C and 8E.

In some embodiments of the present technology, the sample swab 7450 may have visible markings or indicia 7110a, 7110b, which the user may use to determine whether the sample swab 7450 has been inserted into a proper position in the sample chamber 7418. For example, the indicia 7110a may indicate the standby position, and the indicia 711b may indicate the crush position. In some embodiments, the indicia 7110a, 7110b may have different colors for easy identification of one from the other.

In some embodiments of the present technology, the apparatus 7410 may comprise a second chamber 7422 disposed in the housing 7412. The second chamber 7422 may be separated from the sample chamber 7418 by a breakable first seal 7424 that may cover or block a conduit 7425 between the second chamber 7422 and the sample chamber 7418. In some embodiments, the second chamber 7422 may contain a diluent fluid 7421. In some embodiments, when the sample swab 7450 is moved in the second direction X2, the liner 7426 may move in the second direction X2 together with the sample swab 7450. In some embodiments, the first seal 7424 may be attached to the liner 7426 or may be part of the liner 7426, such that movement of the liner 7426 in the second direction X2 may pull the first seal 7424 away from covering the conduit 7425 and thus cause the seal 7424 to break. That is, the first seal 7424 may be pulled in the second direction X2 along with the liner 7426 and may unblock the conduit 7425. Breakage of the first seal 7424 may allow the diluent fluid 7421 to enter the sample chamber 7418 via the conduit 7425. In some embodiments, the diluent fluid 7421 may combine with the sample fluid 7419 to form a diluted sample fluid 7423 in the sample chamber 7418.

According to some embodiments of the present technology, the diagnostic device 7410 may comprise a third chamber 7428 disposed in the housing 7412. The third chamber 7428 may be separated from the sample chamber 7418 by a breakable second seal (not shown) that may cover a conduit (not shown) between the sample chamber 7418 and the third chamber 7428. In some embodiments, the third chamber 7428 may be a test chamber 7428 and may contain a LFA strip 7432 configured to detect a presence of one or more pathogen(s). As with the first seal 7424, the second seal may be attached to the liner 7426 or may be part of the liner 7426, such that movement of the liner 7426 in the second direction X2 may break the second seal. In some embodiments, breakage of the second seal may enable the diluted sample fluid 7423 to flow into the third chamber 7428 and reach the LFA strip 7432.

FIG. 8B schematically depicts a sectional view of the housing 7412 in a plane through a line identified by 8B-8B in FIG. 8A, according to some embodiments of the present technology. In some embodiments, a base end of the liner 7426 may be sandwiched between an outer ring R1′ and an inner ring R2′. The outer ring R1′ may be configured to cover and seal the conduit 7425 between the sample chamber 7418 and the second chamber 7422, and may form at least a part of the first seal 7424 separating the sample chamber 7418 from the second chamber 7422. The outer ring R1′ also may be configured to cover and seal the conduit between the sample chamber 7418 and the third chamber 7428, and may form at least part of the second seal separating the sample chamber 7418 from the third chamber 7428. When the sample swab 7450 is moved in the second direction X2, the seal portion 7454 of the sample swab 7450 may pull the liner 7426 in the second direction X2, thus causing the outer ring R1′ to be pulled in the second direction X2 away from the base 7412b of the housing 7412. Movement of the outer ring R1′ away from the base 7412b of the housing 7412 may cause the first seal 7424 to break (e.g., to move away from a sealing position), to enable the sample chamber 7418 and the second chamber 7422 to be in fluid communication with each other via the conduit 7425. Similarly, movement of the outer ring R1′ away from the base 7412b of the housing 7412 may cause the second seal to break, to enable the sample chamber 7418 and the third chamber 7428 to be in fluid communication with each other. As noted above, breakage of the first seal 7424 and/or the second seal may comprise movement of the outer ring R1′ and need not involve ripping or tearing of material.

According to some embodiments of the present technology, the inner ring R2′ may be used to position the liner 7426 in the sample chamber 7418. In some embodiments, a lower edge 7426a of the liner 7426 may abut against the inner ring R2′ when the liner 7426 is properly inserted in the sample chamber 7418, as depicted in FIG. 8A.

According to some embodiments of the present technology, the sample swab 7450 may trigger a mechanism that causes movement of the crusher 7416 without the sample swab 7450 exerting a force on the crusher 7416. In some embodiments, a portion of the sample swab 7450 may trigger or activate a mechanism that causes the crusher 7416 to move against and rupture the capsule 7415. For example, during insertion of the sample swab 7450 in the sample chamber 7418, a portion of the sample swab 7450 may contact a release mechanism that enables the crusher 7416 to rupture the capsule 7415.

2.3 Sample Swabs Configured to Move or Trigger Movable Crushers

As discussed above, the crusher 7416 may be configured to move in the first chamber 7414 by direct or indirect contact with the contact portion 7452 of the sample swab 7450. FIG. 7 depicts a perspective view of the sample swab 7450, according to some embodiments of the present technology. In some embodiments, the indirect contact may involve the sample swab 7450 triggering a mechanism that causes movement of the crusher 7416 without exerting a moving force on the crusher 7416.

The sample swab 7450 may be comprised of an elongate handle 7455. The swab element 7456 may extend from an end of the handle 7455 via a stem 7457. In some embodiments, the seal portion 7454 of the sample swab 7450 may encircle a portion of the handle 7455 near the swab element 7456, as shown in FIG. 7. The contact portion 7452 may be a tab that extends laterally from the handle 7455. The housing 7412 may be comprised of a slot 7412c configured to receive the contact portion 7452 when the sample swab 7450 is inserted in the sample chamber 7418. As depicted in FIGS. 8A and 8B, the slot 7412c may extend into the housing 7412 as a channel 7427 in which the contact portion 7452 may slide in the first and second directions X1, X2. Although the contact portion 7452 is depicted to contact the crusher 7416 directly in FIGS. 8C and 8D, it should be understood that the contact portion 7452 may instead engage with another object that in turn pushes the crusher 7416 when the sample swab 7450 is moved in the first direction X1 to, e.g., crush the capsule 7415 and deliver the sample into the sample chamber 7418. As will be appreciated, the slot 7412c and the channel 7427 may be configured to ensure that the contact portion 7452 contacts the crusher (directly or indirectly), to causes the crusher 7416 to crush the capsule 7415. Also, as will be appreciated, a portion of the liner 7426 may have a channel (not shown) configured to permit movement of the contact portion 7452 during movement of the sample swab 7450 in the first and second directions X1, X2.

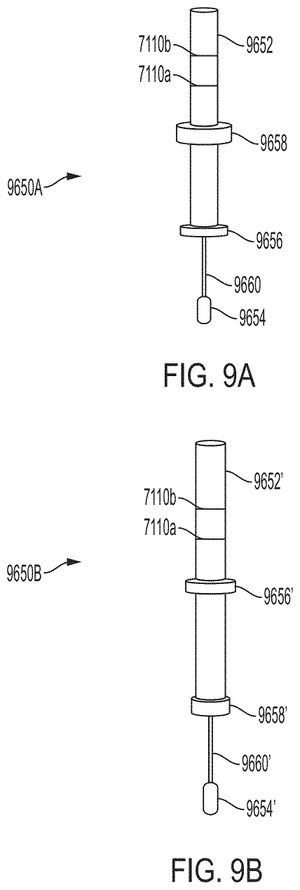

As will be appreciated, a sample swab may have different configurations for causing movement of the crusher 7416. FIG. 9A depicts a crush-capable sample swab 9650A, according to some embodiments of the present technology. The sample swab 9650A may be comprised of an elongate handle 9652. A swab element 9654 may extend from an end of the handle 9652 via a stem 9660. In some embodiments, a seal portion 9656 of the sample swab 650A may encircle a portion of the handle 652 near the swab element 9654. A contact portion 9658 may encircle and extend laterally from the handle 9652 and may be configured to contact a crusher (e.g., the crusher 7416) directly or indirectly when the sample swab 9650A is inserted in a sample chamber. In some embodiments, the contact portion 9658 may be positioned upstream on the handle 9652 relative to the seal portion 9656 and may have an outer diameter that is greater than an outer diameter of the seal portion 9656. The diameter of the contact portion 9658 may be sufficient for the contact portion 9658 to contact the crusher directly, to move the crusher, or to contact a movable object configured to move the crusher. In some embodiments, the seal portion 9656 may be configured to form a movable seal with the sample chamber or with a movable liner forming an inner surface of the sample chamber. The sample chamber and/or the liner may be comprised of a seal section having a diameter configured to form the movable seal with them seal portion 9656 of the sample swab 9650A, and also may be comprised of a crush section having a diameter configured to accommodate movement of the contact portion 9658.

FIG. 9B depicts a crush-capable sample swab 9650B that is a variation of the sample swab 9650A, according to some embodiments of the present technology. The sample swab 9650B may be comprised of an elongate handle 9652′. A swab element 9654′ may extend from an end of the handle 652′ via a stem 9660′. A contact portion 9658′ may extend laterally from the handle 9652′ and may be configured to contact a crusher (e.g., the crusher 7416) directly or indirectly when the sample swab 9650B is inserted in a sample chamber. In some embodiments, the contact portion 9658′ may encircle a portion of the handle 9652′ near the swab element 9654′. A seal portion 9656′ of the sample swab 9650B may encircle the handle 9652 and may be configured to form a movable seal with the sample chamber or with a movable liner forming an inner surface of the sample chamber. In some embodiments, the seal portion 9656′ may be positioned upstream from the contact portion 9658′ and may have an outer diameter that is greater than an outer diameter of the contact portion 9658′. With such a configuration, the contact portion 9658′ may be within a sealed volume of the sample chamber when the seal portion 9656′ forms a seal with the sample chamber of with the liner in the sample chamber. The diameter of the contact portion 9658′ may be sufficient for the contact portion 9658′ to contact the crusher directly, to move the crusher, or to contact a movable object configured to move the crusher.

### 3. Test Methodologies

The diagnostic devices described herein may be used to detect whether a test subject is afflicted with a communicable disease by detecting whether a target nucleic-acid sequence corresponding to a pathogen of interest and indicative of the disease is present in a sample obtained from the test subject, which may be a human subject, a non-human animal subject, a plant subject, a fungus subject, or a subject comprised of environmental material (e.g., a soil sample, a dust sample, etc.). The sample may be comprised of, for example, any one or any combination of saliva, blood, feces, urine, and mucus obtained from the test subject, and/or may be cells obtained from the test subject by other means (e.g., by scraping the test subject's skin, by cutting/plucking hairs from the test subject, etc.). Target nucleic-acid sequences and techniques that may be used for their detection are described below.

Target nucleic-acid sequences may be associated with a variety of diseases or disorders. In some embodiments of the present technology, the diagnostic devices described herein may be used to diagnose at least one disease or disorder caused by a pathogen. In some embodiments, the diagnostic devices may be configured to detect a nucleic acid encoding a protein (e.g., a nucleocapsid protein) of SARS-CoV-2, which is the virus that causes COVID-19. In some embodiments, the diagnostic devices may be configured to identify particular strains of a pathogen (e.g., a virus). In some embodiments, a diagnostic device may utilize and be comprised of an assay vehicle (e.g., an LFA strip) comprised of a first test line configured to detect a nucleic-acid sequence of SARS-CoV-2 and a second test line configured to detect a nucleic-acid sequence of a SARS-CoV-2 virus having a D614G mutation (i.e., a mutation of the 614^{th} amino acid from aspartic acid (D) to glycine (G)) in its spike protein. In some embodiments, one or more target nucleic-acid sequences may be associated with a single-nucleotide polymorphism (SNP). In certain cases, the diagnostic devices may be used for rapid genotyping to detect whether a SNP, which may affect medical treatment, is present.

In some embodiments of the present technology, the diagnostic devices described herein may be configured to diagnose two or more diseases or disorders. This may be referred to herein as multiplexed testing. In certain cases, for example, a diagnostic device may utilize and be comprised of an LFA strip comprised of a first test line configured to detect a nucleic-acid sequence of SARS-CoV-2, a second test line configured to detect a nucleic-acid sequence of an influenza virus (e.g., an influenza A virus), and a third line configured to detect a nucleic-acid sequence of another influenza virus (e.g., an influenza B virus) or a nucleic acid sequence of a bacterium.

3.1 Lysis of Samples

According to some embodiments of the present technology, lysis may be performed on a sample by chemical lysis techniques (e.g., exposing the sample to one or more lysis reagents) and/or thermal lysis techniques (e.g., heating the sample). In chemical lysis, lysis may be performed by one or more lysis reagents, discussed below.

According to some embodiments of the present technology, a lysis reagent may be in solid form (e.g., lyophilized, dried, crystallized, air jetted, etc.). For example, a solid lysis reagent may be in the form of a pellet, or capsule, or gelcap, or tablet. In some embodiments, a solid lysis reagent may be included in a caged cap, as described above. In some embodiments, a lysis reagent may be comprised of one or more additional reagents (e.g., a reagent to reduce or eliminate cross contamination).

According to some embodiments of the present technology, a solid lysis reagent may be shelf stable for a relatively long period of time. In some embodiments, a lysis pellet, or capsule, or gelcap, or tablet may be shelf stable for at least 1 month, at least 3 months, at least 6 months, at least 1 year, at least 5 years, or at least 10 years. In some embodiments, a solid lysis reagent may be thermostabilized and may be stable across a wide range of temperatures. In some embodiments, a lysis pellet, or capsule, or gelcap, or tablet may be stable at a temperature of at least 0° C., at least 10° C., at least 20° C., at least 37° C., at least 65° C., or at least 100° C. As will be appreciated, a solid lysis reagent may be activated before or during use with a sample by contact with a buffer fluid.

As noted above, thermal lysis may be accomplished by applying heat to a sample. According to some embodiments of the present technology, thermal lysis may be performed by applying a lysis heating protocol comprised of heating the sample at one or more temperatures for one or more time periods or durations using any suitable heater (e.g., the heater 960).

3.2 Nucleic-Acid Amplification

Following lysis, one or more target nucleic acids (e.g., a nucleic acid of a target pathogen) may be amplified, according to some embodiments of the present technology. In some embodiments, DNA may be amplified according to any nucleic-acid amplification method known in the art. For example, nucleic-acid amplification methods that may be employed may include isothermal amplification methods, which include: loop-mediated isothermal amplification (LAMP), recombinase polymerase amplification (RPA), nicking enzyme amplification reaction (NEAR), thermophilic helicase dependent amplification (tHDA), nucleic acid sequence-based amplification (NASBA), strand displacement amplification (SDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), whole genome amplification (WGA), and CRISPR-related amplification, such as CRISPR-Cas9-triggered nicking endonuclease-mediated strand displacement amplification (CRISDA). In some embodiments, an isothermal amplification method that may be performed in a test procedure may be comprised of applying heat to a sample. For example, heat may be applied to a sample fluid containing the sample. In some embodiments, the isothermal amplification method may be comprised of applying an amplification heating protocol, which may be comprised of heating the sample at one or more temperatures for one or more time periods using any appropriate heater (e.g., the heater 960).

In embodiments where a target pathogen may have RNA as its genetic material, the target pathogen's RNA may need to be reverse transcribed to DNA prior to amplification.

3.3 Molecular Switches

As described herein, a sample may undergo lysis and amplification prior to detection of a target nucleic-acid sequence. Reagents associated with lysis and/or amplification may be in solid form (e.g., lyophilized, dried, crystallized, air jetted, etc.). According to some embodiments of the present technology, one or more (and, in some cases, all) of the reagents necessary for lysis and/or amplification may be present in a single pellet, capsule, gelcap, or tablet. In some embodiments, the pellet, capsule, gelcap, or tablet may be comprised of two or more enzymes, and it may be necessary for the enzymes to be activated in a particular order. Therefore, in some embodiments, the enzyme-containing tablet, pellet, capsule, or gelcap may further be comprised of one or more molecular switches.

Molecular switches, as used or described herein, may be molecules that, in response to certain conditions, reversibly switch between two or more stable states. According to some embodiments of the present technology, a condition that causes a molecular switch to change its configuration may be associated with any one or any combination of: pH, light, temperature, an electric current, microenvironment, and presence of ions and/or other ligands. In some embodiments, the condition may be heat. In some embodiments, the molecular switches may be comprised of aptamers. Aptamers may refer generally to oligonucleotides or peptides that may bind to specific target molecules (e.g., the enzymes described herein). The aptamers, upon exposure to heat or other conditions, may dissociate from the enzymes. With use of molecular switches, one or more of the processes described herein (e.g., lysis, decontamination, reverse transcription, amplification, etc.) may be performed in a single test tube with a single enzymatic tablet, pellet, capsule, or gelcap.

3.4 CRISPR/Cas Techniques

According to some embodiments of the present technology, CRISPR/Cas detection techniques may be used to detect a target nucleic-acid sequence. For example, one or more CRISPR/Cas detection reagents may be included on an LFA strip. CRISPR generally may refer to Clustered Regularly Interspaced Short Palindromic Repeats, and Cas generally may refer to a particular family of proteins. In some embodiments, a CRISPR/Cas detection platform or technique may be combined with an isothermal amplification method to create a single-step reaction (Joung et al., “Point-of-care testing for COVID-19 using SHERLOCK diagnostics,” 2020). For example, amplification and CRISPR detection may be performed using reagents having compatible chemistries (e.g., reagents that do not interact detrimentally with one another and are sufficiently active to perform amplification and detection). In some embodiments, CRISPR/Cas detection may be combined with LAMP.

### 4. Reagents

According to some embodiments of the present technology, the diagnostic devices described herein may comprise and/or utilize reagents (e.g., lysis reagents, nucleic-acid amplification reagents, CRISPR/Cas detection reagents, and the like) in various test procedures of a diagnostic test. In some embodiments, one or more of the reagents may be contained within a diagnostic device (e.g., in a reaction vial of the diagnostic device). In some embodiments, one or more of the reagents may be provided separately (e.g., in one or more caged caps, in one or more separate vials, etc.). For example, a diagnostic device may be comprised of one or more caged caps comprising one or more lysing reagents and/or one or more amplification reagents.

According to some embodiments of the present technology, at least one (and, in some instances, each) of the reagents used in a diagnostic test may be in liquid form (e.g., in solution). In some embodiments, at least one (and, in some instances, each) of the reagents used in a diagnostic test may be in solid form (e.g., lyophilized, dried, crystallized, air jetted, and the like) and may be activated with buffer fluids prior to or during use.

4.1 Lysing Reagents

According to some embodiments of the present technology, the reagents may be comprised of one or more lysis reagents. A lysis reagent may refer generally to a reagent that promotes cell lysis either alone or in combination with one or more other reagents and/or one or more conditions (e.g., heating). In some embodiments, the lysis reagents may be comprised of one or more enzymes. Non-limiting examples of suitable enzymes may include lysozyme, lysostaphin, zymolase, cellulose, protease, and glycanase. In some embodiments, the lysis reagent(s) may be comprised of one or more detergents. Non-limiting examples of suitable detergents may include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the lysis reagents may be comprised of an RNase inhibitor (e.g., a murine RNase inhibitor). In some embodiments, a concentration of the RNase inhibitor may be is at least 0.1 U/μL, at least 1.0 U/μL, or at least 2.0 U/μL. In some embodiments, the concentration of the RNase inhibitor may be in a range from 0.1 U/μL to 0.5 U/μL, 0.1 U/μL to 1.5 U/μL, or 1.0 U/μL to 2.0 U/μL. In some embodiments, the lysis reagents may comprise Tween (e.g., Tween 20, Tween 80).

4.2 Contamination-Prevention Reagents According to some embodiments of the present technology, the reagents may be comprised of at least one reagent that works to reduce or eliminate potential carryover contamination from prior tests (e.g., prior tests conducted with a common apparatus and/or in a same area). In some embodiments, the reagents may be comprised of thermolabile uracil DNA glycosylase (UDG). In some embodiments, UDG may prevent carryover contamination from prior tests by degrading products that have already been amplified (i.e., amplicons) while leaving unamplified samples untouched and ready for amplification. In some embodiments, a concentration of UDG may be at least 0.01 U/μL, at least 0.03 U/μL, or at least 0.05 U/μL. In some embodiments, the concentration of UDG may be in a range from 0.01 U/μL to 0.02 U/μL or 0.01 U/μL to 0.04 U/μL.

4.3 Reverse Transcription Reagents

According to some embodiments of the present technology, the reagents may be comprised of one or more reverse transcription reagents. As noted above, a target pathogen may have RNA as its genetic material, which may need to be reverse transcribed to DNA prior to amplification. In some embodiments, the reverse transcription reagents may facilitate such reverse transcription. In some embodiments, the reverse transcription reagents may be comprised of a reverse transcriptase, a DNA-dependent polymerase, and/or a ribonuclease (RNase). A reverse transcriptase may refer generally to an enzyme that transcribes RNA to complementary DNA (cDNA) by polymerizing deoxyribonucleotide triphosphates (dNTPs). An RNase may refer generally to an enzyme that catalyzes the degradation of RNA. In some embodiments, an RNase may be used to digest RNA from an RNA-DNA hybrid.

4.4 Nucleic-Acid Amplification Reagents

According to some embodiments of the present technology, the reagents may comprise one or more nucleic-acid amplification reagents. In some embodiments, the nucleic-acid amplification reagents may comprise LAMP reagents, RPA reagents, and NEAR reagents, known in the art. In some embodiments, an enzyme (e.g., Bsm DNA polymerase) may serve as an amplification reagent.

4.5 Reagent Stability Enhancers

According to some embodiments of the present technology, the reagents may comprise one or more additives that may enhance reagent stability (e.g., protein stability). Non-limiting examples of suitable additives may include trehalose, polyethylene glycol (PEG), polyvinyl alcohol (PVA), and glycerol.

4.6 Buffers

According to some embodiments of the present technology, the reagents may comprise one or more reaction buffers. Non-limiting examples of suitable buffers may include phosphate-buffered saline (PBS) and Tris. In some embodiments, the buffers may be buffer fluids. In some embodiments, the buffers may have a relatively neutral pH. In some embodiments, the buffers may have a pH in a range from 5.0 to 7.0, 6.0 to 8.0, 7.0 to 9.0, or 8.0 to 9.0. In some embodiments, the buffers may comprise one or more salts. Non-limiting examples of suitable salts may include magnesium acetate tetrahydrate, potassium acetate, and potassium chloride. In some embodiments, the buffers may comprise Tween (e.g., Tween 20, Tween 80). In some embodiments, the buffers may comprise an RNase inhibitor. In some embodiments, Tween and/or an RNase inhibitor may facilitate cell lysis. In a particular, non-limiting embodiment of the present technology, the buffers may comprise 25 mM Tris buffer, 5% (w/v) poly(ethylene glycol) 35,000 kDa, 14 mM magnesium acetate tetrahydrate, 100 mM potassium acetate, and greater than 85% volume nuclease free water.

### 5. Detection Devices

As noted above, according to some embodiments of the present technology, LFA strips (e.g., the LFA strip 1014, 7432) may be used as assay vehicles to test for whether a target nucleic-acid sequence, corresponding to a pathogen of interest, is present in a sample obtained from a user. In some embodiments, the target nucleic acid-acid sequence may be amplified (i.e., amplicons) prior to detection via an LFA strip. In some embodiments, an LFA strip may provide results that may be read or interpreted in a non-clinical setting by a lay person (e.g., a person not trained in laboratory procedures). LFA strips may be comprised of reagents or substances for indicating the presence (or absence) of a target nucleic-acid sequence. In some embodiments, an LFA strip may be configured to detect two or more different target nucleic-acid sequences.

According to some embodiments of the present technology, an LFA strip useable with the diagnostic devices described herein may be comprised of one or more fluid-transporting layers, which may be comprised of one or more absorbent materials that allow a fluidic sample to move from one end of the LFA strip (e.g., an intake end) to an opposite end of the LFA strip. In some embodiments, fluid movement may be via wicking or capillary action. Non-limiting examples of suitable materials may include polyethersulfone, cellulose, polycarbonate, nitrocellulose, sintered polyethylene, and glass fibers.

According to some embodiments of the present technology, an LFA strip may be comprised of a plurality of sub-regions. In some embodiments, the fluidic sample may be introduced to a first sub-region (e.g., a region in contact with a sample pad) and may subsequently flow through a second sub-region (e.g., a particle conjugate pad) comprised of a plurality of labeled particles. In some embodiments, the particles may be comprised of gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some embodiments, as an amplicon-containing fluidic sample flows through the second sub-region, a labeled nanoparticle may bind to a label of an amplicon, thereby forming a particle-amplicon conjugate. In some embodiments, the fluidic sample may subsequently flow through a third sub-region comprised of one or more test lines. In some embodiments, a first test line may be comprised of a capture reagent (e.g., an immobilized antibody) configured to detect a first target nucleic-acid sequence. In some embodiments, a particle-amplicon conjugate may be captured by one or more capture reagents (e.g., immobilized antibodies), and an opaque marking may appear on the first test line. In some embodiments, the LFA strip may comprise one or more additional test lines configured to detect one or more different target nucleic-acid sequences. In some embodiments, the third sub-region of the LFA strip may further comprise one or more control lines. For example, a control line may be a human (or animal) nucleic-acid control line configured to detect a nucleic acid (e.g., RNase P) that is generally present in all humans (or animals). The control line may be used to confirm whether a human (or animal) sample was successfully collected, nucleic-acid sequences from the sample were amplified, and the amplicons were transported through the LFA strip successfully.

According to some embodiments of the present technology, a diagnostic device may be comprised of two or more LFA strips arranged in parallel, such that a sample fluid may flow in each LFA strip independently of the other LFA strip(s).

### 6. Test Kits

According to some embodiments of the present technology, the diagnostic devices described herein may be part of a test kit useable by a lay person, i.e., a person who is not trained in medical and/or laboratory techniques or procedures. The test kit may be a stand-alone test kit that does not require the use of additional laboratory equipment to perform a diagnostic test. In some embodiments, the test kit may be comprised of a swab device (e.g., the sample swab 1100, 7450, 9650A, 9650B) and a diagnostic device (e.g., the diagnostic device 1000, 7410). One or more reagents necessary for the diagnostic test may be provided in the diagnostic device itself (e.g., in a burstable capsule held in a cavity of the diagnostic device, in a portion of the diagnostic device confined by rupturable seals, etc.) or may be provided in a reagent carrier (e.g., a caged cap) to be added by a user during a test procedure.

6.1 Heater

According to some embodiments of the present technology, a heater may be provided as part of a diagnostic device. For example, as shown in FIG. 8A the heater 7460 may be provided as part of the diagnostic device 7410 to heat a sample solution (e.g., for lysis and/or amplification). In some embodiments, the heater may be a printed circuit board (PCB) heater. For example, the PCB heater may be comprised of a bonded PCB with a microcontroller, thermistors, and/or resistive heating elements. In some embodiments, the heater may be pre-programmed with one or more heating protocols. For example, the heater may be pre-programmed with a lysis heating protocol and/or an amplification heating protocol. The lysis heating protocol may be a set of one or more temperatures and one or more time periods that facilitate lysis of a sample. The amplification heating protocol may be a set of one or more temperatures and one or more time periods that facilitate amplification of a nucleic-acid sequence. In some embodiments, the heater may be comprised of an auto-start mechanism that performs heating according to a pre-programmed temperature profile needed for lysis and/or amplification upon activation of the auto-start mechanism by a user.

6.2 Instructions & Software

According to some embodiments of the present technology, a test kit may be comprised instructions associated with sample collection and/or operation of a diagnostic device. For example, the instructions may be comprised of directions for handling a swab device to obtain a sample from a subject as well as directions for providing a collected sample to a diagnostic device (or a component thereof) for further processing. The instructions may be provided in any form readable by a user. For example, the instructions may be written or published, verbal, audible (e.g., telephonic), digital, optical, visual (e.g., videotape, DVD, etc.), and/or provided via electronic communications (including Internet or web-based communications). In some embodiments, the instructions may combine graphical information with textual information. In some embodiments, the instructions may be provided as part of a software-based application.

According to some embodiments of the present technology, the instructions may be provided as part of a software-based application that may be downloaded to a smartphone or other type of portable electronic device, and contents of the downloaded application may guide a user through steps to use a diagnostic device and/or to perform test procedures of a diagnostic test. In some embodiments, the instructions may instruct a user when to add certain reagents and how to do so.

According to some embodiments of the present technology, a software-based application may be connected (e.g., via a wired or wireless connection) a diagnostic device to control the diagnostic device or components thereof and/or to read and analyze test results. In some embodiments, the application may be configured to process an image of an LFA strip captured by an imaging device (e.g., a smartphone camera, etc.) and to evaluate the image to provide a positive or negative test result for each of one or more test lines on the LFA strip.

It should be understood that the features and details described above may be used, separately or together in any combination, in any of the embodiments discussed herein.

Some aspects of the present technology may be embodied as one or more methods. Acts performed as part of a method may be ordered in any suitable way. Accordingly, embodiments may be constructed in which acts may be performed in an order different than described or illustrated, which may include performing some acts simultaneously, even though they may be shown or described as sequential acts in illustrative embodiments.

Aspects described in one embodiment may be combined in any manner with aspects described in other embodiments.

Any use of ordinal terms such as “first,” “second,” “third,” etc., in the description and the claims to modify an element does not by itself connote any priority, precedence, or order of one element over another, or the temporal order in which acts of a method are performed, but is or are used merely as labels to distinguish one element or act having a certain name from another element or act having a same name (but for use of the ordinal term) to distinguish the elements or acts.

The indefinite articles “a” and “an,” as used herein in the specification and in the claims, unless clearly indicated to the contrary, should be understood to mean “at least one.”

Any use herein, in the specification and in the claims, of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Any use herein, in the specification and in the claims, of the phrase “equal” or “the same” in reference to two values (e.g., distances, widths, etc.) should be understood to mean that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. As used herein in the specification and in the claims, the term “or” should be understood to have the same meaning as “and/or” as defined above.

The terms “approximately” and “about” if used herein may be construed to mean within ±20% of a target value in some embodiments, within ±10% of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

What is claimed is:

1. A rapid diagnostic test apparatus, comprising:
a sample chamber;
a fluid chamber connected to the sample chamber by a conduit; and
a test chamber separated from the sample chamber by a breakable first seal.

2. The apparatus of claim 1, further comprising:
a movable liner forming a portion of an inner surface of the sample chamber.

3. The apparatus of claim 2, further comprising:
a sample swab configured to be inserted into an opening of the sample chamber and to seal the sample chamber with a slidable seal.

4. The apparatus of claim 3, wherein the sample swab is comprised of:
a swab element, and
a handle comprised of a seal portion configured to slide along the inner surface of the sample chamber to slidably seal of an internal cavity of the sample chamber.

5. The apparatus of claim 4, wherein the seal portion of the handle is comprised of a gasket configured to press against and slide along the inner surface of the sample chamber to slidably seal the sample chamber.

6. The apparatus of claim 5, further comprising:
a burstable capsule containing a first fluid,
wherein the sample swab is configured to cause the capsule to burst when the sample swab is inserted in the sample chamber to an inserted position, such that the first fluid is permitted to form a sample solution in the sample chamber by interaction with a sample carried by the swab element of the sample swab.

7. The apparatus of claim 6, wherein:
the sample swab is configured to be moved in a first direction to insert the swab element of the sample swab into the sample chamber, and
the sample swab is configured to be moved from an inserted position in the sample chamber in a second direction, different from the first direction, to break the first seal.

8. The apparatus of claim 7, wherein the liner is configured to move with the sample swab in the second direction to break the first seal.

9. The apparatus of claim 8, wherein:
the fluid chamber is separated from the sample chamber by a breakable second seal,
the liner is configured to move with the sample swab in the second direction to break the second seal, and
the liner is configured to break the second seal before breaking the first seal.

10. The apparatus of claim 9, wherein the sample swab is comprised of a handle with markings indicating at least one insertion position of the sample swab when the sample swab is inserted in the sample chamber.

11. The apparatus of claim 6, wherein the sample swab is configured to extend into the internal cavity of the sample chamber to contact and burst the capsule.

12. The apparatus of claim 6, wherein:
the capsule is disposed in the fluid chamber,
the fluid chamber is comprised of a crusher configured to move in the fluid chamber, and
the crusher is configured to be moved by a contact portion of the sample swab to burst the capsule, when the sample swab is inserted to the inserted position in the sample chamber.

13. The apparatus of claim 6, wherein:
the test chamber is comprised of a lateral-flow assay (LFA) strip, and
when movement of the sample swab causes the first seal to break, the sample solution in the sample chamber is permitted to enter the test chamber.

14. The apparatus of claim 6, further comprising a heater configured to heat the sample chamber.

15. A rapid diagnostic test apparatus, comprising:
a first chamber;
a second chamber separated from the first chamber by a breakable first seal;
a third chamber separated from the first chamber by a breakable second seal; and
a movable liner forming a portion of an inner surface of the first chamber, wherein the liner is comprised of at least a portion of one of the first and second seals or at least a portion of both of the first and second seals.

16. The apparatus of claim 15, wherein the first seal and the second seal are connected to each other.

17. The apparatus of claim 15, further comprising:
a sample swab configured to be inserted into an opening of the first chamber and to seal the first chamber with a slidable seal.

18. The apparatus of claim 17, wherein the sample swab is comprised of:
a swab element, and
a handle comprised of a seal portion configured to slide along the inner surface of the first chamber to slidably seal an internal cavity of the first chamber.

19. The apparatus of claim 18, wherein the seal portion of the handle of the sample swab is configured to slide along the liner to slidably seal the internal cavity of the first chamber when the swab element is inserted into the first chamber.

20. The apparatus of claim 18, wherein:
the sample swab is configured to be moved in a first direction to insert the swab element of the sample swab into the first chamber, and
when the sample swab is in an inserted position in the first chamber, the sample swab is configured to be moved in a second direction, different from the first direction, to break one of or both of the first and second seals.
